(19) United States
(12) Patent Application Publication
(10) Pub. No.: US 2026/0128170 A1
(43) Pub. Date: May 7, 2026

(54) BIOMOLECULAR DETECTION METHOD AND DETECTION SYSTEM THEREOF

(71) Applicant: National Cheng Kung University, Tainan City (TW)

(72) Inventors: Jiunn-Der LIAO, Tainan City (TW); Han LEE, Tainan City (TW); Kuan-Hung LIU, Tainan City (TW)

(21) Appl. No.: 19/382,625

(22) Filed: Nov. 7, 2025

Related U.S. Application Data
(60) Provisional application No. 63/717,494, filed on Nov. 7, 2024.

(51) Int. Cl. G16H 50/20; B82Y 15/00; G01N 21/65; G01N 33/543; G01N 33/68; G16B 40/10; G16B 40/20; G16H 50/70

(52) U.S. Cl. CPC G16H 50/20; G01N 21/658; G01N 33/54346; G01N 33/6812; G16B 40/10; G16B 40/20; G16H 50/70; B82Y 15/00; G01N 2201/129; G01N 2333/165

(57) ABSTRACT

A biomolecular detection method is disclosed. The method includes following steps: providing a surface-enhanced Raman spectroscopy (SERS) substrate; applying a biological sample onto the SERS substrate, wherein the biological sample is a medical specimen; acquiring a SERS signal on the SERS substrate through using a SERS spectrometer; and applying a trained machine learning model of a detection system to analyze the full spectral range of the SERS signal, or one or more key feature regions of the full spectral range, and output a biomolecular detection result of the biological sample.

### CROSS-REFERENCE TO RELATED APPLICATIONS

This application claims the benefit of filing date of U.S. Provisional Application Ser. No. 63/717,494, entitled “A method that combines label-free SERS spectroscopy and artificial intelligence assistance to accurately detect the outer membrane of specific pathogens or biomedical molecular structures” filed Nov. 7, 2024 under 35 USC § 119 (e)(1).

### BACKGROUND OF THE INVENTION

### Field

The present application relates to a detection method and detection system, and particularly to a detection method and detection system for biomolecules.

### Description of Related Art

Currently, the most commonly used biomolecular detection method (e.g. PCR (Polymerase chain reaction), ELISA (enzyme linked immunosorbent assay) and LC-MS (Liquid chromatography mass spectrometry)) is labeling detection, which has high accuracy and strong identification ability. However, most of these methods require “labels” for detection, such as antibodies, enzymes, or probes. These testing procedures are not only complex and time-consuming, but also require expensive equipment and professional operating people, making them unsuitable for scenarios requiring rapid response, such as clinical settings or telemedicine.

Surface-enhanced Raman spectroscopy (Hereinafter referred to as SERS) is a technique that can directly detect molecular signals, theoretically without the need for labels, and with very high sensitivity. However, in practical applications, the uneven distribution of “hot spots” on the nanomaterials can lead to unstable and difficult-to-reproduce signals, further affecting the reliability of the diagnosis. In addition, while machine learning (such as deep learning) can help analyze spectral data, current machine learning methods often require significant analysis time and are not easily adapted to clinical equipment. Due to technological limitations, spectral analysis still faces many challenges in applications such as rapid diagnosis and remote health monitoring.

Therefore, a novel biomolecular detection method and system combining SERS and artificial intelligence is needed to improve the above-mentioned problems.

### SUMMARY OF THE INVENTION

One objective of the present application is to provide a biomolecular detection method. The biomolecular detection method, comprises following steps: providing a SERS substrate; applying a biological sample onto the SERS substrate, wherein the biological sample is a medical specimen; acquiring a SERS signal on the SERS substrate through using a SERS spectrometer; and using a trained machine learning model of a detection system to analyze a full spectral range of the SERS signal, or one or more key feature regions of the full spectral range, and output a biomolecular detection result of the biological sample.

Another objective of the present application is to provide a detection system, adapted for a biomolecular detection method including providing a SERS substrate; applying a biological sample onto the SERS substrate, wherein the biological sample is a medical specimen; acquiring a SERS signal on the SERS substrate through using a SERS spectrometer; and using a trained machine learning model of a detection system to analyze a full spectral range of the SERS signal, or one or more key feature regions of the full spectral range, and output a biomolecular detection result of the biological sample, comprising: a signal preprocessing unit to perform a signal preprocessing procedure on the SERS signal; and a trained machine learning model to analyze the preprocessed SERS signals.

Other novel features of the disclosure will become more apparent from the following detailed description when taken in conjunction with the accompanying drawings.

### BRIEF DESCRIPTION OF DRAWINGS

FIG. 1 is a flowchart of the steps of a biomolecular detection method combining SERS and artificial intelligence according to an embodiment of the present application.

FIG. 2 is a system architecture diagram of a detection system according to an embodiment of the present application.

FIG. 3 is a flowchart of the signal preprocessing procedure of an embodiment of the present application.

FIG. 4 is a flowchart of the key feature search method of an embodiment of the present application.

FIG. 5 is a flowchart of the detection method of the first example of the present application.

FIG. 6 is a flowchart of the training process of the trained machine learning model in the first example of the present application.

FIG. 7 is a flowchart of the detection method of the second example of the present application.

FIG. 8 is a flowchart of the training process of the trained machine learning model in the second example of the present application.

### DETAILED DESCRIPTION OF THE INVENTION

The exemplary embodiments of the present application will now be described in detail with reference to the accompanying drawings. Whenever possible, identical component symbols are used in diagrams and descriptions to represent the same or similar parts.

The present application uses certain terms to refer to specific components, but in practice, the same component may be referred to by different names. Furthermore, unless otherwise defined, all terms used herein (including technical and scientific terms) have the same meaning as commonly understood by one of ordinary skill in the art to which this application pertains. Furthermore, in this application, words such as “containing,” “including,” and “comprising” are open-ended terms, and therefore should be interpreted as “containing but not limited to . . . ”. The terms “approximately,” “substantially,” or “roughly” are generally interpreted as being within 10% of a given value or range, or within 5%, 3%, 2%, 1%, or 0.5% of a given value or range.

The ordinal numbers used in this application, such as “first” and “second,” to modify elements do not imply or represent any prior ordinal number of that (or those) element, nor do they represent the order of one element with another, or the order of manufacturing methods.

Furthermore, descriptions such as “when . . . ” or “ . . . time” in this application indicate states such as “now, before, or after”, and are not limited to situations where they occur simultaneously. Descriptions such as “set on . . . ” in this application indicate the corresponding positional relationship between two elements and do not limit whether the two elements are in contact, unless otherwise specified. Furthermore, when this application describes multiple effects, the use of the word “or” between the effects indicates that the effects can exist independently, but does not preclude the possibility that multiple effects can exist simultaneously.

In this document, the term “electrical connection” or “coupled connection” includes various direct and indirect electrical connection methods, such as direct contact between two parties to transmit electrical signals, or the transmission of electrical signals between two parties through a third or more intermediaries. In this article, “mutual communication” can include data transmission between the two parties via wired or wireless communication. In this article, the term “information” may refer to, for example, the content obtained through data analysis or the content after data transformation, and is not limited to thereto. Furthermore, in this application, the term “adjacent” is used to describe objects that are close to each other, and adjacent objects may or may not be in contact.

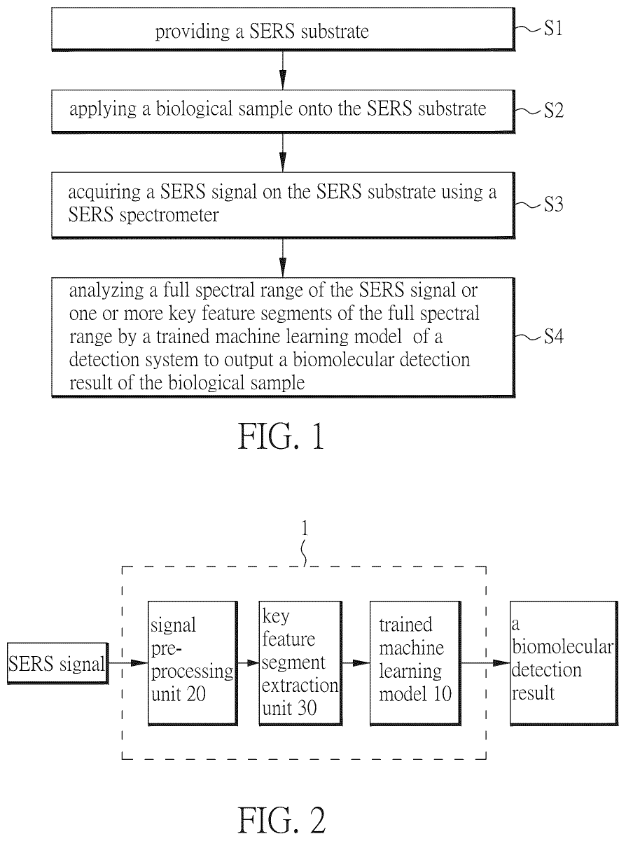

FIG. 1 is a flowchart of the steps of a biomolecular detection method combining SERS and artificial intelligence according to an embodiment of the present application (hereinafter referred to as the detection method). As shown in FIG. 1, step S1 is first performed to provide a SERS substrate. Next, step S2 is performed to apply a biological sample onto the SERS substrate, wherein the biological sample is a medical specimen. Then, step S3 is performed to acquire a SERS signal on the SERS substrate using a SERS spectrometer. Subsequently, step S4 is executed, where a trained machine learning model 10 of a detection system 1 (as shown in FIG. 2) analyzes a full spectral range of the SERS signal or one or more key feature segments of the full spectral range to output a biomolecular detection result of the biological sample.

In one embodiment, the type of biological sample may include a viral envelope protein or a middle molecule toxin (or called “a medium molecular weight molecule toxin”, e.g. medium molecular weight molecule in uremic toxin”), and the biomolecular detection result includes a viral morphology detection result or a concentration or morphology detection result of a target molecule. In one embodiment, the source of the biological sample includes nasopharyngeal swabs, throat swabs, plasma, urine, saliva, or blood samples. In one embodiment, the detection method can be applied to the identification of outer membrane proteins of a pathogen and the typing of variant strains, or to the real-time monitoring of middle molecule toxin concentrations associated with kidney disease (e.g. chronic kidney disease), and is not limited thereto.

In one embodiment, the SERS substrate includes a bulk material and a plurality of gold nanoparticles (Au NPs) or a plurality of silver nanoparticles (Ag NPs), wherein the plurality of gold nanoparticles or silver nanoparticles covers the substrate bulk material. Furthermore, the substrate bulk material can be a fiber structure or a bowl-shaped structure. The following examples all use fibrous structures. In one embodiment, the diameter of each gold or silver nanoparticle is between 1 and 100 nanometers (nm) in diameter, 1-5 μm in length (of the fiber structure is between 1 and 5 micrometers (μm), and the diameter of the fiber structure is between 20 and 200 nanometers. Furthermore, when the biological sample is applied to the SERS substrate, the SERS substrate can directly adsorb the pathogenic outer membrane or biomolecules of the biological sample. Therefore, the SERS signal obtained from the SERS substrate will be beneficial for the analysis of the detection system 1.

In one embodiment, the full spectral range may, for example, be between 600 and 1800 cm⁻¹. In one embodiment, the one or more key feature segments are spectral segments corresponding to viral mutation sites, tryptophan (Trp) residue regions, or specific biotoxins, but are not limited to these.

Please refer to FIG. 1 and FIG. 2. FIG. 2 is a system architecture diagram of a detection system 1 according to an embodiment of the present application. As shown in FIG. 2, the detection system 1 includes the trained machine learning model 10 and a signal preprocessing unit 20, which is electrically or communicatively connected to the trained machine learning model 10. The signal preprocessing unit 20 is used to perform a signal preprocessing procedure on the SERS signal obtained in step S3, so as to convert the SERS signal from the raw signal into a more stable and more favorable preprocessed SERS signal for analysis. The trained machine learning model 10 is used to analyze the full spectral range or one or more key feature regions of the preprocessed SERS signal, and then output the biomolecule detection result.

In addition, the detection system 1 may selectively include a key feature segment extraction unit 30, which may be electrically or communicatively connected to the signal preprocessing unit 20 and the trained machine learning model 10, respectively, or the key feature segment extraction unit 30 may be integrated into the trained machine learning model 10. The key feature segment extraction unit 30 can be used to extract the data corresponding to one or more key feature segments from the full spectral range of the preprocessed SERS signal, and then passes them to the trained machine learning model 10 for analysis. In one embodiment, the key feature segment extraction unit 30 can be configured, for example, to select the segment to be extracted, but is not limited thereto. Therefore, when the trained machine learning model 10 needs to analyze one or more key feature segments of the full spectral range of the SERS signal, the detection system 1 can have a key feature segment extraction unit 30. Moreover, when the trained machine learning model 10 analyzes the full spectral range of the SERS signal, the detection system 1 may not have the key feature segment extraction unit 30, or the operation of the key feature segment extraction unit 30 may be paused.

Next, the details of the detection system 1 will be explained.

Regarding the detection system 1: In one embodiment, the detection system 1 may include, for example, one or more processors, or one or more electronic devices equipped with processors, such as, but not limited to, a computer. In another embodiment, the detection system 1 may also be, for example, one or more software or firmware programs, and is not limited thereto. The trained machine learning model 10, the signal preprocessing unit 20, or the key feature segment extraction unit 30 may be, for example, a functional module. The functionality of the functional module can be achieved by a processor executing one or more instructions from a computer program, which can be, for example, software or firmware, and can be stored in a non-transitory computer-readable medium. The non-transitory computer-readable media can be, for example, memory, hard drive, optical disc, USB flash drive, or cloud drive, and is not limited thereto. Furthermore, the trained machine learning model 10, the signal preprocessing unit 20, or the key feature segment extraction unit 30 can be implemented by the same or different processors, or by the same or different electronic devices.

Regarding the trained machine learning model 10: In one embodiment, the type of the trained machine learning model 10 may include, but is not limited to, a support vector machine (SVM), a random forest model, a K-nearest neighbor (KNN) model, a convolutional neural network (CNN), a kernel model, an ensemble learning model, or a transformer architecture model. In one embodiment, the trained machine learning model 10 is formed by training an initial model in a training phase. That is, the initial model needs to be trained and iterated through multiple training data to obtain the ability to analyze unknown SERS signals and output the biomolecular detection results of the unknown SERS signals, thus forming the trained machine learning model 10. In one embodiment, the initial model is trained using supervised learning, meaning each training data has a label associated with the biomolecule detection result. Furthermore, in one embodiment, each training data is, for example, a SERS signal, and the biomolecule detection result associated with that SERS signal is known information.

Regarding the signal preprocessing unit 20: Please refer to FIG. 1, FIG. 2 and FIG. 3. FIG. 3 is a flowchart of the signal preprocessing procedure of an embodiment of the present application. The signal preprocessing procedure may include steps S31 to S35. It should be noted that, as long as it is reasonable and feasible, steps S31 to S35 can be deleted or their order can be changed according to actual needs.

As shown in FIG. 3, step S31 is executed first. The signal preprocessing unit 20 performs a spike removal process on the SERS signal acquired in step S3. For example, the signal preprocessing unit 20 can use Whitaker-Haye's modified z-scores method to remove abnormal spikes or cosmic spikes in the SERS signal, and is not limited thereto.

Next, step S32 is executed. The signal preprocessing unit 20 performs a baseline correction on the SERS signal to remove background values from the SERS signal. For example, the signal preprocessing unit 20 can use the asymmetric least squares (ALS) method to perform baseline correction, but is not limited thereto.

Next, step S33 is executed, in which the signal preprocessing unit 20 performs a smoothing process on the SERS signal. For example, the signal preprocessing unit 20 may use the Savitzky-Golay smoothing method to smooth the signal, but is not limited thereto.

Next, step S34 can be executed, whereby the signal preprocessing unit 20 performs a normalization process on the SERS signal. For example, the signal preprocessing unit 20 can use a min-max normalization method (e.g., normalizing all signal values to the range [0,1]) or a z-scores normalization method to perform normalization, or it can use a machine learning model specifically for normalization, and is not limited thereto. Thus, the steps of the signal preprocessing procedure can be understood. In addition, in one embodiment, between step S33 and step S34, an outlier removal process may be performed, for example, by using Hotelling's T² or Q-residual analysis to remove outliers from the SERS signal, and is not limited thereto.

Regarding the extraction unit 3 for this key feature segment. In one embodiment, the key feature segment extraction unit 30 can extract segments based on predetermined content (e.g., the segments to be extracted have been predefined). However, the decision of which segments should be extracted is not necessarily a required function of the key feature segment extraction unit 30. In one embodiment, the decision of which segments to extract can be made by performing a key feature search method, some of which can be performed manually or by machines. If the operation is performed by a machine, the machine may use key feature segment extraction unit 30 or other equipment for that key feature segment, but is not limited thereto.

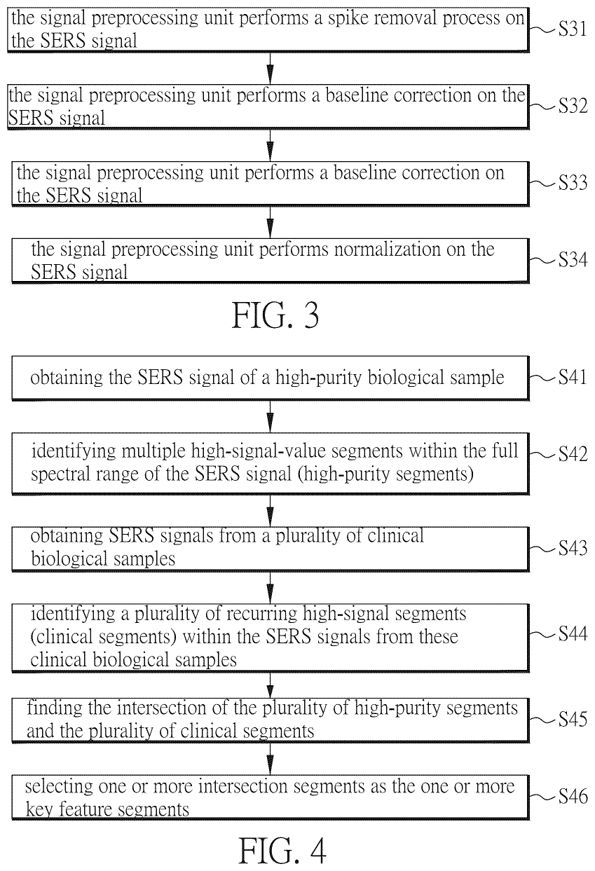

Please refer to FIG. 1 to FIG. 4. FIG. 4 is a flowchart of the key feature search method of an embodiment of the present application. First, step S41 is performed to obtain the SERS signal of a high-purity biological sample. The process of obtaining the SERS signal can be referred to steps S1 to S3 in FIG. 1. Next, step S42 is performed to identify multiple high-signal-value segments within the full spectral range of the SERS signal from the high-purity biological sample; these segments are referred to below as high-purity segments. Next, step S43 is executed to obtain SERS signals from a plurality of clinical biological samples. Then, step S44 is executed to identify, within the SERS signals from these clinical biological samples, a plurality of recurring high-signal segments, hereinafter referred to as clinical segments. Next, step S45 is executed, which involves finding the intersection of the plurality of high-purity segments and the plurality of clinical segments. If the difference between the upper and lower limits of the two segments is less than 30%, 20%, 10%, or 5% respectively, then the two segments can be selected, with the larger of the two segments being the primary selection, but not limited to these two categories. Next, step S46 is executed, which selects one or more intersection segments as the one or more key feature segments. Thus, the key feature search method can be understood. Furthermore, in other embodiments, methods such as PLS-DA can also be used to search key feature segments.

Generally speaking, the SERS signal from a high-purity biological sample can reflect more features. Moreover, the recurrence of these features in SERS signals from clinical biological samples indicates that these features possess stable properties. Therefore, it is suitable as the basis for analysis of the trained machine learning model 10. Therefore, the key feature search method can be understood.

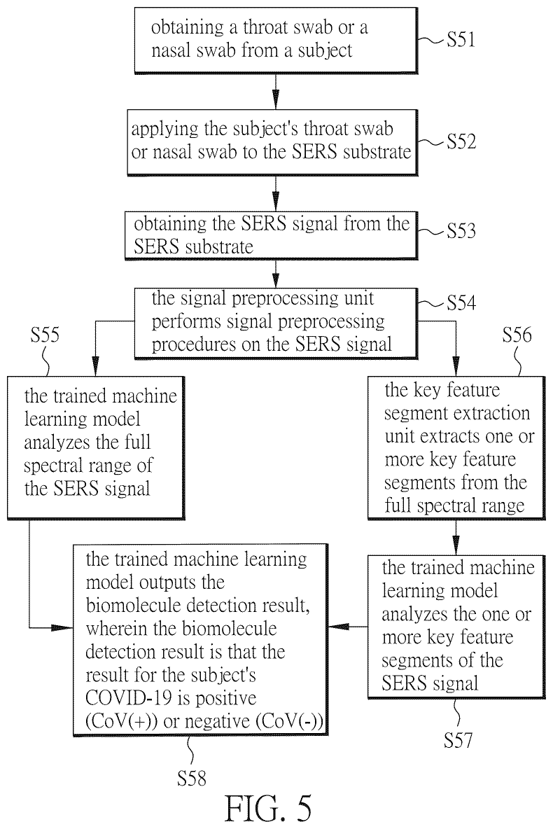

Please refer to FIG. 1 to FIG. 5. FIG. 5 is a flowchart of the detection method of the first example of the present application, mainly used for virus morphology detection. For clarity, the first example uses a positive (CoV(+)) or negative (CoV(−)) test result for COVID-19 as the target.

As shown in FIG. 5, step S51 is executed first. First, step S51 is performed, obtaining a throat swab or a nasal swab from a subject's throat or nasal cavity. Next, step S52 is performed, applying the subject's throat swab or nasal swab to the SERS substrate. Next, step S53 is executed to obtain the SERS signal from the SERS substrate. Next, step S54 is executed, whereby the signal preprocessing unit 20 performs signal preprocessing procedures on the SERS signal. Next, step S55 is executed, and the trained machine learning model 10 analyzes the full spectral range of the SERS signal. Alternatively, steps S56 and S57 are performed, whereby the key feature segment extraction unit 30 extracts one or more key feature segments from the full spectral range, and then the trained machine learning model 10 analyzes the one or more key feature segments of the SERS signal. Next, step S58 is executed, and the trained machine learning model 10 outputs the biomolecule detection result, where the biomolecule detection result is, for example, the test result for the subject's COVID-19 is positive (CoV(+)) or negative (CoV(−)), which belongs to binary classification.

The details of steps S51 to S52 are as follows: Nasopharyngeal and throat swab specimens were collected from inpatients at National Cheng Kung University Hospital (Tainan, Taiwan), under the Institutional Review Board approval (IRB number: B-ER-112-006). Classification of specimens through RT-PCR was also done to determine specimen class, whether CoV (+) or CoV(−). Prior to the acquisition of SERS spectra, specimens were stored in universal transport medium (UTM) until needed.

Prior to dropping the specimen onto the SERS substrate, as a pre-treatment process, specimens were passed through a 220 nm filter and then centrifuged at 500 g to minimize non-target species that are larger than the average SARS-COV-2 virion size.

Furthermore, Two types of Au—ZrO2 hybrid SERS substrates used throughout this study: one features Au nanoparticles (Au NPs) upon the surface of bowl-like nanostructures formed by ZrO2, denoted as Au NPs/pZrO2 and the other features Au NPs upon a multilayer nanofibrous ZrO2 structure referred to as Au NPs/fZrO2. Au NPs/pZrO2 substrates were formed through a template-assisted process with the use of polystyrene nanoparticles (PS NPs) forming a monolayer onto Si substrates through evaporative self-assembly. ZrO2 precursor with a gel-like consistency made with ZrCl4 and isopropanol was then deposited onto the PS NPs-coated Si substrates through spin coating then subjected to thermal treatment at 600° C. for 3 h, simultaneously burning off the PS NPs while forming the ZrO2, resulting in bowl-like pores. After which, Au NPs were then deposited onto the bowl-like porous ZrO2 through thermal evaporation, resulting in clusters of irregularly shaped Au NPs.

On the other hand, Au NPs/fZrO2 were fabricated using the same ZrO2 precursor mentioned earlier, and spin coated onto a Si wafer for 30 s at 5000 rpm at room temperature before subjecting to the same thermal treatment as mentioned. After thermal treatment, samples were allowed to cool in the furnace down to room temperature and were taken out, ready to be used.

The fabrication of Au NPs/fZrO2 is somehow similar to the fabrication of Au NPs/pZrO2 except that PS NPs were not involved. Instead, ZrO2 precursor was simply spin-coated onto a pre-cleaned substrate at 5000 rpm for 30 s at ambient temperature before subjecting to calcination at 600° C. for 3 h. After which, Au NPs were coated upon the substrates through an electron beam evaporator (VT1-10CE, UL VAC Inc., Japan) with a thickness of 1.5 nm and a rate of 0.1 A/s under 7×106 torr.

Moreover, silver nanoparticles (Ag NPs) also can be used for the detection method of the present application. The details of the synthesis of silver nanoparticles (Ag NPs) applied in the method of the present application is illustrated below: the synthesis of silver nanoparticles (Ag NPs) was carried out using sodium borohydride (NaBH₄) as the primary reducing agent and trisodium citrate (TSC) as both a secondary reducing agent and stabilizer. The reduction reaction was performed at two different temperatures (60° C. and 90° C.) to control the nanoparticle formation process. To begin, a freshly prepared aqueous solution containing NaBH₄ and TSC was vigorously stirred in the dark while heated to 60° C. for 30 min to ensure homogeneity. After this period, a dropwise addition of AgNO₃ solution was performed, followed by an increase in temperature to 90° C. Once the reaction mixture reached 90° C., the pH was adjusted to 10.5 using 0.1 M NaOH, and heating continued for another 20 min until a noticeable color change was observed, indicating nanoparticle formation.

After synthesis, the nanoparticle suspension was allowed to cool to room temperature. To remove unreacted reducing agents and stabilize the Ag NPs, the solution was centrifuged at 12,000 rpm for 15 min, washed three times with deionized water, and then redispersed in deionized water. To minimize interference from surface-adsorbed impurities on the Raman signal, surface modification of Ag NPs was performed by introducing potassium iodide (KI). First, 5 mL of sodium citrate-reduced Ag NPs suspension was centrifuged at 6,500 rpm for 20 min at 20° C., and the supernatant was discarded. The concentrated nanoparticle pellet (25 μL) was then mixed with 25 μL of 1 mM KI solution and allowed to react at room temperature for 60 min, ensuring complete interaction between I⁻ ions and the Ag NP surface.

After iodide modification, 5 μL of dichloromethane (DCM) was added to the mixture, followed by 10 μL of 0.01 M Ca²⁺ solution (CaCl₂)·2H₂O). The modified silver nanoparticles (mAg NPs) were thoroughly mixed and stored in the dark for subsequent SERS detection.

First, pure β2-m and leptin were characterized by Raman signal analysis to identify the Raman-active vibrational modes of both molecules. Clinical dialysate samples were then collected at 0-, 15-, and 240-min during hemodialysis for comparative analysis. SERS spectra revealed distinct β2-m characteristic peaks at 896, 1340-1360, 1463, and 1674 cm−1, while a transient peak at 1384 cm−1 indicated dynamic molecular interactions during dialysis. This detection system enhances the Raman signal due to the synergistic plasmonic coupling between Au NPs (on the substrate) and mAg NPs (near the periphery of the target molecule), generating localized electromagnetic hotspots. This combination of detection systems improves signal reproducibility and detection sensitivity even in complex clinical matrices.

Swab specimens were completely thawed first before dropping onto the fabricated SERS substrates mounted on an acrylic holder, then covered with a covered slip to avoid direct contact between the instrument and the liquid sample. A portable Raman spectrometer (Single Laser Micro Raman Spectroscopy, NS220, Nanoscope Systems, Inc., Korea) with a spectral resolution of ±10 cm 1 and equipped 633 nm laser with a spot size diameter of 2 μm, was used to acquire SERS spectra of the specimens; measurement was done in a level 2 plus biosafety laboratory. A minimum of 10 spectra were collected from each clinical specimen, taken from different locations throughout the substrate. After collection of raw data, spectra designated for training data were assigned accordingly based on specimen class.

For steps S53 to S55 and S57 to S58, please refer to the illustration in the preceding paragraphs, and therefore will not be described here further. In one embodiment, in step S56, the X-axis of a full spectrum of the SERS signal corresponds to the Raman Shift (i.e., the full spectral range), with units of cm⁻¹, while the Y-axis corresponds to the concentration of pathogen outer membrane proteins on the throat swab or nasal swab. In one embodiment, when the biomolecule originates from a nasal swab and the carrier comprises Au NPs/pZrO₂, the one or more key feature segments may, for example, comprise nine segments, namely 870-882 cm⁻¹, 902-920 cm⁻¹, 1100-1116 cm⁻¹, 1170-1188 cm⁻¹, 1239-1252 cm⁻¹, 1300-1315 cm⁻¹, 1374-1389 cm⁻¹, 1455-1470 cm⁻¹ and 1572-1590 cm⁻¹, and not limited thereto. In one embodiment, when the biomolecule originates from a throat swab and the carrier contains Au NPs/fZrO₂, the one or more key feature segments may, for example, comprise seven segments, namely 876, 920, 1115, 1172, 1250, 1306, 1381, 1460, 1585 cm⁻¹, and not limited thereto. The above values are merely examples and not limitations.

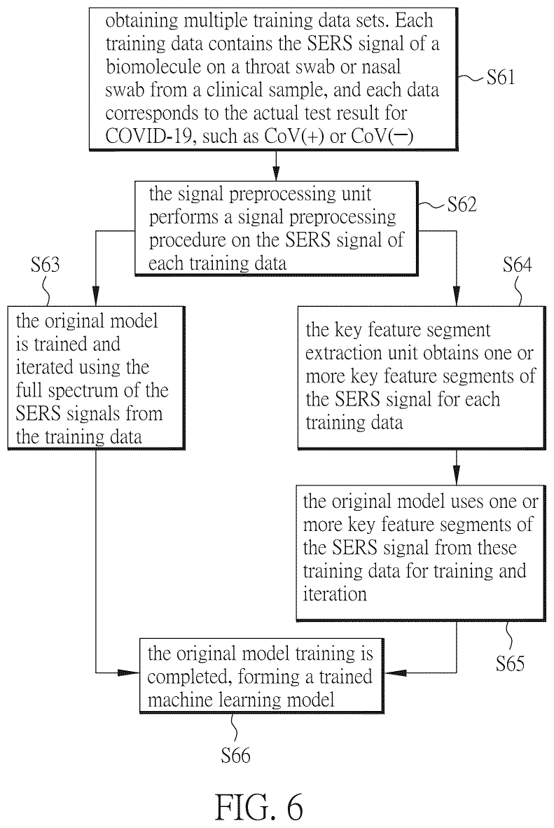

In the first example, the machine learning model 10 has been trained to have the ability to analyze the characteristics of SERS signals to determine whether COVID-19 is positive (CoV(+)) or negative (CoV(−)). The training process is described below. FIG. 6 is a flowchart of the training process of the trained machine learning model 10 in the first example of the present application. Please also refer to FIGS. 1 to 5.

As shown in FIG. 6, firstly, step S61 is executed to obtain multiple training data sets. Each training data set contains the SERS signal of a biomolecule on a throat swab or nasal swab from a clinical sample, and each data set also contains a label. This label corresponds to the actual test result for COVID-19, such as CoV(+) or CoV(−). In one embodiment, a portion of the clinical samples are used as the training group (e.g., 259 records.), a portion of the clinical samples are used as the test group (e.g., 12 records.), and another portion of the clinical samples are used as the blind test group (e.g., 6 tests (3 positive and 3 negative) and 6 records. (2 positive and 4 negative)). In addition, the training data corresponding to positive results is, for example, 120 records, and the training data corresponding to negative results is, for example, 120 records. The above values are merely examples and not limitations. Moreover, the process of obtaining these training materials is applicable to the descriptions of steps S51 to S53 above, and therefore will not be elaborated further.

Next, step S62 is executed, and the signal preprocessing unit 20 performs a signal preprocessing procedure on the SERS signal of each training data. Next, step S63 is executed, and the original model is trained and iterated using the full spectrum of the SERS signals from the training data. Alternatively, steps S64 and S65 are executed, and the key feature segment extraction unit 30 obtains one or more key feature segments of the SERS signal for each training data. Moreover, the original model uses one or more key feature segments of the SERS signal from these training data for training and iteration. Next, step S66 is executed, and the original model training is completed, forming a trained machine learning model 10. Thus, the training process of the trained machine learning model 10 in the first example can be understood.

Examples of experimental results from this application and conventional chemical analysis methods are described in tables 1 and 2 below. As shown in the table below, the prediction results of the present application are superior to those of conventional analysis methods.

TABLE 1

| | accuracy | sensitivity |
|---|---|---|
| AuNPs/pZrO₂ or AuNPs/fZrO₂ | 50 | 100 |
| conventional chemical analysis/ | | |
| Throat | | |
| AuNPs/pZrO₂/throat swab/KNN | 33.3 | 50 |
| model | | |
| AuNPs/fZrO₂/throat swab/KNN | 50 | 100 |
| model | | |

TABLE 2

| | accuracy | sensitivity |
|---|---|---|
| AuNPs/pZrO₂ or AuNPs/fZrO₂ | 66.6 | 100 |
| conventional chemical analysis/ | | |
| nasal cavity | | |
| AuNPs/pZrO₂/Nasopharyngeal | 41.7 | 60 |
| swab/KNN model | | |
| AuNPs/fZrO₂/Nasopharyngeal | 50 | 80 |
| swab/KNN model | | |

Furthermore, for experimental results regarding more parameter variations, please refer to the contents of the US provisional application.

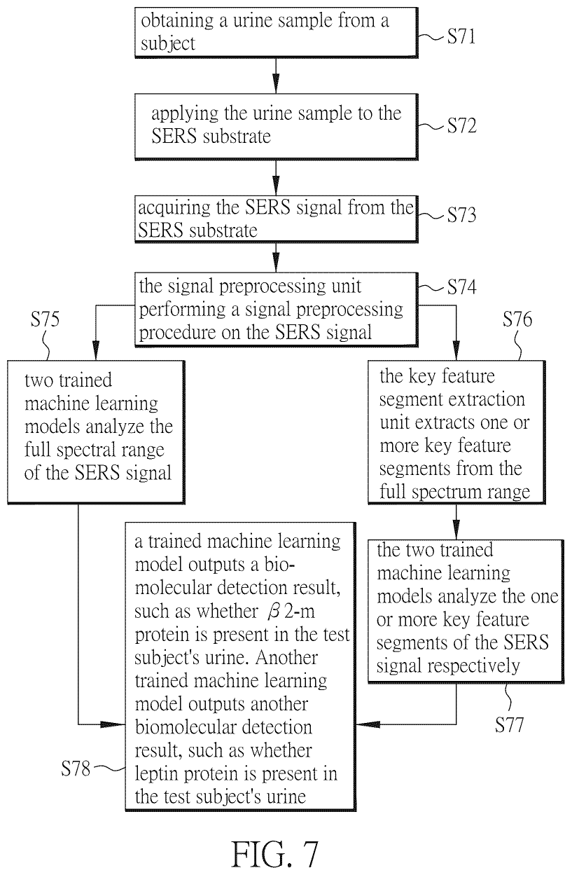

FIG. 7 is a flowchart of the detection method of the second example of the present application, mainly used for the detection results of the concentration or form of the target molecule. Please also refer to FIGS. 1 to 6. For ease of explanation, the second example aims to detect the presence of β2-m protein or leptin protein in a subject's urine. The presence of β2-m protein or leptin protein in urine indicates a higher likelihood that the individual has kidney disease.

As shown in FIG. 7, first step S71 is performed to obtain a urine sample from a subject. Then step S72 is performed to apply the urine sample to the SERS substrate. Next, step S73 is executed to acquire the SERS signal from the SERS substrate. Then, step S74 is executed, where the signal preprocessing unit 20 performs a signal preprocessing procedure on the SERS signal. Next, step S75 is executed, in which two trained machine learning models 10 analyze the full spectral range of the SERS signal. Alternatively, steps S76 and S77 are executed, whereby the key feature segment extraction unit 30 extracts one or more key feature segments from the full spectrum range, and then the two trained machine learning models 10 analyze the one or more key feature segments of the SERS signal respectively. Next, step S78 is executed, in which a trained machine learning model 10 outputs a biomolecular detection result, such as whether β2-m protein is present in the test subject's urine. Another trained machine learning model 10 outputs another biomolecular detection result, such as whether leptin protein is present in the test subject's urine.

The details of steps S71 to S72 are similar to the details of steps S51 to S52. Similarly, Two types of Au—ZrO2 hybrid SERS substrates used throughout this study: one features Au nanoparticles (Au NPs) upon the surface of bowl-like nanostructures formed by ZrO2, denoted as Au NPs/pZrO2 and the other features Au NPs upon a multilayer nanofibrous ZrO2 structure referred to as Au NPs/fZrO2. Moreover, the procedure for further applying specimen onto the SERS substrates is similar to that illustrated in step S51 to S52.

Regarding steps S73 to S78, the details of these steps are generally applicable to the explanation of the first example, so the following explanation mainly focuses on the differences. In the second example, the detection method uses two trained machine learning models 10 for analysis, one model to analyze the presence of β2-m protein and the other model to analyze the presence of leptin protein. Furthermore, in step S76, the one or more key feature segments may, for example, include 7 segments, namely 500-550, 760, 932, 1012, 1243, 1299, 1340-1360, 1384, 1580 cm−1, 1620 cm−1, and is not limited thereto. The above values are merely examples and not limitations.

The SERS peaks detected for β2-m and leptin is by way of using different nanoparticle configurations (such as Au NPs/fZrO₂, Ag NPs+Au NPs/fZrO₂, and mAg NPs+Au NPs/fZrO₂). For β2-m, a total of 10 characteristic peaks were identified, categorized as major peaks (highlighted with red and gray bars), minor peaks (green bars), and potential peaks (blue bars). Major peaks include 500-550 cm⁻¹ (S—S bond vibrations), 760 cm⁻¹ (benzene and pyrrole ring breathing modes), 932 cm⁻¹ (C—COO⁻ stretching), and 1012 cm⁻¹ (aromatic ring vibrations), along with peaks at 1243 cm⁻¹, 1580 cm⁻¹, and 1620 cm⁻¹, reflecting functional group dynamics and protein backbone features. Minor peaks, such as 643 cm⁻¹ and 830-850 cm⁻¹, represent tyrosine side-chain vibrations, while potential peaks, including 912 and 959 cm⁻¹, relate to valine C—H stretching and CH₃ bending. These peaks, summarized in Table 1, correspond to specific functional group vibrational modes and were consistently detected across the β-f (II), β-f (III), β-p (II), and β-p (III) configurations, demonstrating the structural analysis capabilities of these SERS-active substrates.

For leptin, 14 characteristic peaks were identified, following a similar color-coded classification with gray dotted lines for major peaks (e.g., 534 cm⁻¹, COOH vibrations; 746 cm⁻¹ and 858 cm⁻¹, phenylalanine symmetric ring breathing; 1454 cm⁻¹, CH₃ asymmetric deformation), green dotted lines for minor peaks (e.g., 1012 cm⁻¹, tryptophan benzene and pyrrole ring breathing), and blue dotted lines for potential peaks (e.g., 793 cm⁻¹, arginine NH₂ rocking). These peaks, as detailed in Table 2, highlight the structural complexity of leptin and the sensitivity of nanoparticle-enhanced SERS detection. Notably, β-f (II), β-f (III), β-p (II), and β-p (III) demonstrated superior enhancement effects for both β2-m and leptin, offering precise detection of functional group vibrational modes critical for molecular analysis.

Note that β2-m major peaks are (1) 550, (2) 760, (3) 932, (4) 1012, (5) 1243, (6) 1299, (7) 1340-1360, (8) 1384, (9) 1580, (10) 1620; and minor peaks are 643, 80-832, 850. Moreover, potential peaks are 912, 959 cm⁻¹. In addition, leptin major peaks are (1) 534, (2) 669, (3) 746, (4) 775, (5) 835, (6) 858, (7) 931, (8) 1003, (9) 1030, (10) 1080, (11) 1240, (12) 1296, (13) 1454, (14) 1624; and minor peaks are 1012, 1620; potential 793, 1129, 1168, 1316, 1445, 1589.

Furthermore, the SERS detection mechanism for β2-m functional groups in a clinical setting, mainly emphasizes the interactions with various nanoparticle configurations. The description highlights β2-m as the primary focus while suggesting the mechanism may also be relevant for leptin. For example, β2-m interacts with Au NPs/fZrO₂, or mAg NP. The hotspots are positioned near the β2-m functional groups, enhancing interactions with Au NP hotspots.

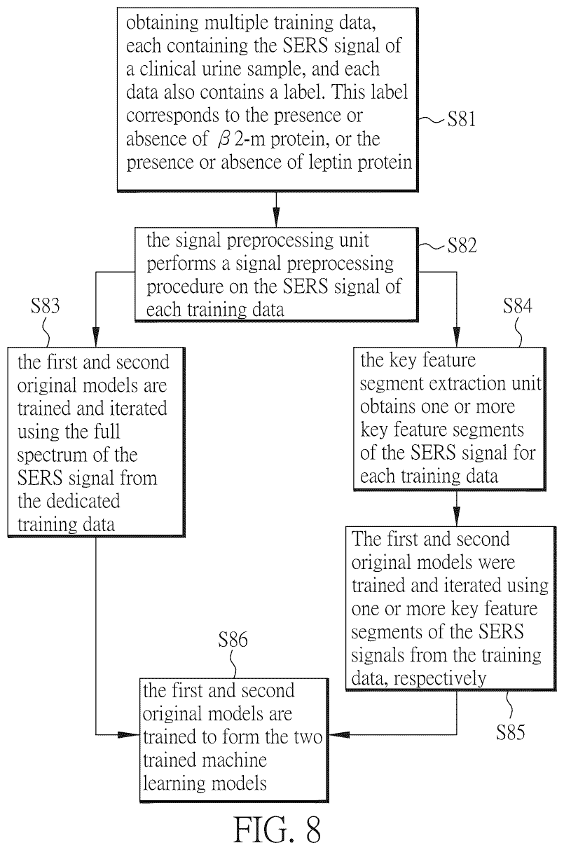

In the second example, the training process of the two trained machine learning models 10 is described as follows. FIG. 8 is a flowchart of the training process for the two trained machine learning models 10 in the second example of the present application. Please also refer to FIGS. 1 to 7.

First, step S81 is executed to obtain multiple training data sets, each containing the SERS signal of a clinical urine sample, and each data set also contains a label. This label corresponds to the presence or absence of β2-m protein, or the presence or absence of leptin protein. It should be noted that β2-m protein and leptin protein may coexist in the same urine sample. Therefore, different labels can be assigned to the training data based on the different output targets of these two trained machine learning models. In addition, for example, 80% of the data is used as the training group, and the remaining 20% is used as the test group. In one embodiment, for example, there are 821 sample data, of which 646 have β2-m protein and 175 do not. These sample data will be provided as training data to the first original model for training. Furthermore, of the 821 sample data, 647 contained the leptin protein, while 174 did not. These sample data will be used as training data for the second original model.

Next, step S82 is executed, and the signal preprocessing unit 20 performs a signal preprocessing procedure on the SERS signal of each training data. Next, step S83 is executed, whereby the first and second original models are trained and iterated using the full spectrum of the SERS signal from the dedicated training data. Alternatively, steps S84 and S85 are executed, and the key feature segment extraction unit 30 obtains one or more key feature segments of the SERS signal for each training data. The first and second original models were trained and iterated using one or more key feature segments of the SERS signals from the training data, respectively. Next, step S86 is executed, and the first and second original models are trained to form the two trained machine learning models 10. Thus, the training process of the trained machine learning model 10 in the second paradigm can be understood.

Examples of experimental results from the present application and conventional chemical analysis methods are described in Tables 3 and 4 below. As shown in the table below, the prediction results of the present application are superior to those of traditional analysis methods.

TABLE 3

| | accuracy | sensitivity |
|---|---|---|
| AuNPs/pZrO₂ or AuNPs/fZrO₂ | 71.4 | 85 |
| conventional chemical analysis/ | | |
| β2-m | | |
| AuNPs/pZrO₂ or AuNPs/fZrO₂ | 82.9 | 100 |
| β2-m/SVM model | | |

TABLE 4

| | accuracy | sensitivity |
|---|---|---|
| AuNPs/pZrO₂ or AuNPs/fZrO₂ | 50 | 85 |
| conventional chemical analysis/ | | |
| leptin | | |
| AuNPs/pZrO₂ or AuNPs/fZrO₂ | 86.1 | 100 |
| leptin SVM model | | |

Furthermore, for experimental results regarding more parameter variations, please refer to the contents of the US provisional application.

In addition, the detection method of the present application can be integrated into a point-of-care testing (POCT) device. The POCT device includes: an automated sample collection module, a sample processing and transport module, a SERS spectral analysis module, a data analysis software platform, a report output module, or a remote clinical decision support system, or the combination thereof. In addition, the detection method of the present application can be applied to: identification of pathogen outer membrane proteins and typing of variant strains, or real-time monitoring of middle molecule toxin concentrations related to kidney disease.

In this way, the problems of the prior art can be solved or improved.

In one embodiment, when a suspected infringing product is discovered, the present application can at least determine whether it falls within the scope of the present application by examining the presence or absence of components, component configuration, mechanical observation, and/or operation mode of the suspected infringing product, and is not limited thereto. Furthermore, if software is involved, the determination can at least be made by analyzing the operation of components or by reverse engineering the operation of the software program.

Details or features between the various embodiments of the present application may be freely mixed and matched as long as they do not violate the spirit of the invention or conflict with it. The above embodiments are merely illustrative examples for ease of explanation. The scope of rights claimed in this application shall be determined by the claims of the patent application, and not limited to the above embodiments.

Although the present disclosure has been explained in relation to its embodiment, it is to be understood that many other possible modifications and variations can be made without departing from the spirit and scope of the disclosure as hereinafter claimed.

1. A biomolecular detection method, comprising following steps:
providing a surface-enhanced Raman spectroscopy (SERS) substrate;
applying a biological sample onto the SERS substrate, wherein the biological sample is a medical specimen;
acquiring a SERS signal on the SERS substrate through using a SERS spectrometer; and
using a trained machine learning model of a detection system to analyze a full spectral range of the SERS signal, or one or more key feature regions of the full spectral range, and output a biomolecular detection result of the biological sample.

2. The biomolecular detection method as claimed in claim 1, wherein a type of the biological sample includes a viral envelope protein or a middle molecule toxin, and the biomolecular detection result includes a viral morphology detection result or a concentration or morphology detection result of a target molecule.

3. The biomolecular detection method as claimed in claim 1, wherein the SERS substrate comprises a bulk material and a plurality of gold nanoparticles (Au NPs) or a plurality of silver nanoparticles (Ag NPs), the plurality of gold nanoparticles or silver nanoparticles cover the substrate bulk material, and the substrate bulk material is of a fiber structure or a bowl-shaped structure.

4. The biomolecular detection method as claimed in claim 3, wherein a diameter of each gold or silver nanoparticle is between 1 and 100 nanometers (nm), a length of the fiber structure is between 1 and 5 micrometers (μm), and a diameter of the fiber structure is between 20 and 200 nanometers.

5. The biomolecular detection method as claimed in claim 3, wherein the biological sample is derived from urine of a test subject, and the SERS substrate is a substrate covered by the plurality of gold nanoparticles, silver nanoparticles, or the combination thereof.

6. The biomolecular detection method as claimed in claim 1, wherein a type of the trained machine learning model comprises a Support Vector Machine (SVM), Random Forest model, K-Nearest Neighbors (KNN) model, Convolutional Neural Network (CNN), Kernel model, ensemble learning model, or transformer architecture model.

7. The biomolecular detection method as claimed in claim 1, wherein the trained machine learning model is formed by training an initial model in a training phase, where training data used in the training phase includes SERS signals from multiple biological samples taken from the throat or nasal cavity of a human body, and each training data has a label that corresponds to either a positive (CoV(+)) or negative (CoV(−)) COVID-19 result.

8. The biomolecular detection method as claimed in claim 1, wherein the trained machine learning model is formed by training an initial model in a training phase, where training data used in the training phase includes SERS signals from multiple biological samples of urine taken from human bodies, and each training data has a label that corresponds to presence or absence of a middle molecule toxin, or to a concentration level of the middle molecule toxin, in which the middle molecule toxin is β2-m protein, or leptin protein.

9. The biomolecular detection method as claimed in claim 1, wherein key feature segments of the full spectral range of the SERS signal correspond to viral mutation sites, tryptophan (Trp) residue regions, or specific biological toxins.

10. The biomolecular detection method as claimed in claim 1, wherein source of the biological sample includes nasopharyngeal swabs, throat swabs, plasma, urine, saliva, or blood samples

11. The biomolecular detection method as claimed in claim 1, wherein the biomolecular detection method is integrated into a point-of-care testing (POCT) device including: an automated sample collection module, a sample processing and transport module, a SERS spectral analysis module, a data analysis software platform, a report output module, or a remote clinical decision support system, or a combination thereof.

12. The biomolecular detection method as claimed in claim 1, wherein the biomolecular detection method is applied to identification of pathogen outer membrane proteins and typing of variant strains, or real-time monitoring of middle molecule toxin concentrations related to kidney disease.

13. A detection system, adapted for a biomolecular detection method including providing a surface-enhanced Raman spectroscopy (SERS) substrate; applying a biological sample onto the SERS substrate, wherein the biological sample is a medical specimen; acquiring a SERS signal on the SERS substrate through using a SERS spectrometer; and using a trained machine learning model of a detection system to analyze a full spectral range of the SERS signal, or one or more key feature regions of the full spectral range, and output a biomolecular detection result of the biological sample, comprising: a signal preprocessing unit to perform a signal preprocessing procedure on the SERS signal; and a trained machine learning model to analyze the preprocessed SERS signals.

14. The detection system as claimed in claim 13, further comprising a key feature segment extraction unit for extracting data corresponding to one or more key feature segments from the full spectral range of the SERS signal after the signal preprocessing procedure.

15. The detection system as claimed in claim 13, wherein the signal preprocessing procedure comprises a sub-procedure for spike removal, baseline correction, smoothing, normalization, or a combination thereof.
